(19) United States
(12) Patent Application Publication
(10) Pub. No.: US 2002/0115707 A1
(43) Pub. Date: Aug. 22, 2002

(54) Process for preparing pure ondansetron hydrochloride dihydrate

(76) Inventors: Rami Lidor-Hadas, Kfar Saba (IL); Eliezer Bachar, Tel Aviv (IL)

Correspondence Address: KENYON & KENYON, ONE BROADWAY, NEW YORK, NY, 10004, US

(21) Appl. No.: 10/045,970

(22) Filed: Jan. 11, 2002

Related U.S. Application Data
(60) Provisional application No. 60/261,051, filed on Jan. 11, 2001.

(51) Int. Cl. A61K 31/403; C07D 209/82

(52) U.S. Cl. 514/411; 548/440

(57) ABSTRACT

An improved method for preparing dimethylamino-methyl-carbazolone and ondansetron base. A recrystallization process for preparing pure ondansetron hydrochloride dihydrate with a purity of at least 99.0% is also disclosed.

### CROSS REFERENCE TO RELATED APPLICATION

This application claims the benefit of Provisional Application Ser. No. 60/261,051, filed Jan. 11, 2001, the disclosure of which is incorporated by reference in its entirety herein.

### FIELD OF THE INVENTION

The present invention relates to an improved process for preparing dimethylamino-methyl-carbazolone. The present invention relates to an improved process for preparing ondansetron base. The present invention also relates to an improved process for recrystallizing ondansetron hydrochloride dihydrate to obtain pure ondansetron hydrochloride dihydrate.

### BACKGROUND OF THE INVENTION

Ondansetron, also known as 1,2,3,9-tetrahydro-9-methyl-3-[(2-methyl-1H-imidazol-1-yl) methyl]-4H-carbazol-4-one is a potent and highly selective serotonin (5-HT₃, 5-hydroxytrptamine receptor 3) antagonist and has the following formula:

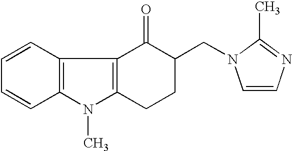

Ondansetron is currently available as an anti-emetic agent, particularly in cancer chemotherapy, and in some other uses such as anti-depressive, anti-migraine and anti-psychotic. It is commonly used in the alleviation of cognitive disorders as in Alzheimer disease, in treatment of rhinitis, psychiatric disorders and for increased vigilance and for control of dependence on narcotics.

U.S. Pat. No. 4,695,578, assigned to the Glaxo Group Limited, describes a process of preparing ondansetron and uses thereof. However, ondansetron prepared according to said process contains impurities and by-products such as 1,2,3,9-tetrahydro-9-methyl-3-methylene-4H-carbazol-4-one.

There is a continuing need for improving the method of preparing ondansetron with high purity that meets the standard for clinical uses.

### OBJECTS AND SUMMARY OF THE INVENTION

The known methods of preparing ondansetron do not achieve a pharmaceutically describe high purity and color. An object of the present invention is to meet a need in the art for a high purity (i.e., at least about 99.0%) and improved color.

Another object of the present invention is to prepare pure ondansetron hydrochloride dihydrate substantially free of any impurities and by-product such as 1,2,3,9-tetrahydro-9-methyl-3-methylene-4H-carbazol-4-one (e.g., the exo-methylene by-product).

Another object of the present invention is to prepare ondansetron hydrochloride dihydrate that has a purity of at least about 99.0%. Preferably, the ondansetron hydrochloride dihydrate has a purity of at least about 99.5%. Most preferably, the ondansetron hydrochloride dihydrate has a purity of at least about 99.9%.

Another object of the present invention is to provide a process for preparing dimethylamino-methyl-carbazolone, the process comprising the steps of:

a) preparing a solution of methyl-carbazolone;

b) heating the solution in the presence of dimethylamino hydrochloride and paraformaldehye;

c) basifying the solution to form a precipitate;

d) separating the precipitate from the solution to obtain dimethylamino-methyl-carbazolone; and

e) drying the dimethylamino-methyl-carbazolone.

Another object of the present invention is a process for preparing ondansetron base, the process comprising the steps of:

a) preparing a solution of methyl-imidzole and dimethylamino-methyl-carbazolone;

b) heating the solution;

c) removing a precipitate containing ondansetron base;

d) washing the precipitate; and

e) drying the precipitate to obtain pure ondansetron base.

Preferably, step e) is followed by recrystallizing the ondansetron base in the presence of activated carbon and methanol.

Another object of the present invention is a process for preparing ondansetron hydrochloride dihydrate, the process comprising the steps of:

a) preparing a solution of ondansetron base;

b) acidifying the solution with hydrogen chloride to form a precipitate;

c) washing the precipiate; and

d) crystallizing ondansetron hydrochloride dihydrate.

### DETAILED DESCRIPTION OF THE INVENTION

As used herein, the term “exo-methylene by-product” refers to 1,2,3,9-tetrahydro-9-methyl-3-methylene-4H-carbazol-4-one. It represents one of the main impurity in ondansetron preparation. Another impurity in ondansetron preparation is dimeric exo-methylene by-product.

Unless otherwise specified, “%” refers to % wt.

As used herein, the term “pure ondansetron” refers to ondansetron that is substantially free of exo-methylene by-product and has a high purity of at least about 99.0%.

As used herein, the term “hydrogen chloride” refers to either a gaseous hydrogen chloride or a solution of hydrogen chloride gas in water.

As used herein, the term “equivalent” refers to molar equivalent.

As used herein, the term “vacuum distillation” refers to the separation of solids from liquids by passing the mixture through a vacuum filter.

As used herein, the term “reflux” refers to during a chemical process, part of the product stream may be returned to the process to assist in giving increased conversion or recovery, as in distillation or liquid-liquid extraction.

As used herein, the term “filter cake” refers to a concentrated solid or semisolid material that is separated from a liquid and remains on the filter after pressure filtration.

The present invention is an improved method of preparing a pure ondansetron hydrochloride dihydrate with purity at least 99.0%. More preferably, the ondansetron hydrochloride dihydrate purity is at least 99.5%. Most preferably, the ondansetron hydrochloride dihydrate purity is at least 99.9%.

The present invention provides an improved method of preparing dimethylamino-methyl-carbazolone. The present invention further provides an improved method of preparing ondansetron base. The present invention further provides an improved method of preparing pure ondansetron hydrochloride dihydrate.

### Preparation of Dimethylamino-Methyl-Carbazolone

The present invention provides a process for preparing dimethyl amino-methyl-carbazolone comprising the steps of:

a) preparing a solution of methyl-carbazolone having the formula:

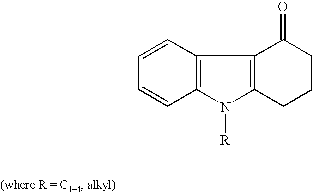

b) heating the solution in the presence of dimethylamino hydrochloride and paraformaldehyde;

c) basifying the solution to form a precipitate;

d) separating the precipitate from the solution to obtain dimethylamino-methyl-carbazolone; and

e) drying the dimethylamino-methyl-carbazolone.

During the heating step, the solution is heated in the presence of dimethylamine hydrochloride and paraformaldehyde in an organic solvent. Preferably, the organic solvent is acetic acid.

Preferably, one equivalent methyl-carbazolone is refluxed with about 1.1 to about 1.5 equivalents of dimethylamine-hydrochloride and paraformaldehyde. Most preferably, one equivalent methyl-carbazolone is refluxed with about 1.2 equivalents of dimethylamnine-hydrochloride and paraformaldehyde. During the heating step, formaldehyde can be used to substitute for paraformaldehyde in the reflux reaction.

Preferably, one equivalent methyl-carbazolone is refluxed with about 4 to about 16 volumes of acetic acid. Most preferably, one equivalent methyl-carbazolone is refluxed with about 4 volumes of acetic acid.

Preferably, the heating step is performed at a temperature of about 70° C. to about 100° C. Most preferably, the heating step is performed at a temperature of about 80° to about 90° C.

Preferably, the heating step is performed for about 6 to about 24 hours. Most preferably, the heating step is performed for about 6 to about 12 hours.

Preferably, the separating step is performed using filtration.

Preferably, the heating step is performed without the use of vacuum distillation or extraction. The heating step performed in the absence of vacuum distillation or extraction consistently yields a better pure dimethylamino-methyl-carbazolone.

The present invention also provides a process of preparing pure dimethylamino-methyl-carbazolone further comprises dissolving the filter cake in acetone and treating with activated carbon and hydrogen chloride.

Preferably, water is added at the basifying step thereafter rendering the solution basic by about 45% sodium hydroxide (NaOH) to a pH range of about 13 to about 14. Preferably, the basifying step is performed in the presence of celite (10%), filter and dry.

Preferably, the dry cake is dissolved in acetone. Preferably, the dissolved cake is treated with activated carbon and hydrogen chloride to precipitate dimethylamino-methyl-carbazolone.

### Preparation of Ondansetron Base

The present invention provides a process for the synthesis of ondansetron base comprising the steps of:

a) preparing a solution of methyl-imidazole and dimethylamino-methyl-carbazolone of the formula

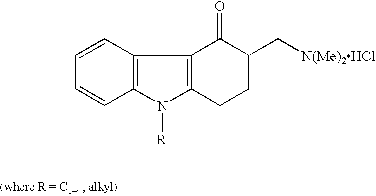

b) heating the solution;

c) removing a precipitate containing containing ondasetron base from the solution;

d) washing the precipitate;

e) drying precipitate to obtain ondansetron base.

The present invention further provides a process for the synthesis of substantially pure ondansetron base, further comprising the steps of:

recrystallizing in the presence of activated carbon and methanol.

During the solution preparation step of methyl-imidazole and dimethylamino-methyl-carbazolone, about 4 to about 6 equivalents methyl-imidazole is preferably added to one equivalent dimethylamino-methyl-carbazolone. Most preferably, about 5 equivalents of methyl-imidazole is added to one equivalent dimethylamino-methyl-carbazolone.

Preferably, the preparation step is performed in the presence of 10% celite.

Preferably, the present invention provides a process for preparing ondansetron base further comprising (after step e) a step of recrystallizing ondansetron base in the presence of activated carbon and methanol.

### Crystallization to Prepare Pure Ondansetron Hydrochloride Dihydrate

The present invention provides an improved method of preparing a pure ondansetron hydrochloride dihydrate. Specifically, the preparation involves crystallization of ondansetron hydrochloride dihydrate from ondansetron base with water and activated carbon and in the absence of an organic solvent.

The crystallization process of the present invention greatly increases the purity of ondansetron hydrochloride dihydrate and dramatically lowers the content of the exo-methylene by-product impurity. Preferably, the crystallization step is performed 1-3 times. Most preferably, the crystallization step is performed two times.

The present invention provides a method of crystallization of ondansetron hydrochloride dihydrate comprising the steps of:

a) preparing a solution of ondansetron base;

b) acidifying the solution with hydrogen chloride to form a precipitate;

c) washing the precipitate; and

d) crystallizing pure ondansetron hydrochloride dihydrate.

Preferably, the solution preparation step is achieved by adding about 3 to about 7 volumes of water to ondansetron base. Most preferably, the solution preparation step is achieved by adding about 5 volumes of water to ondanseton base.

Preferably, the acidifying step is achieved by adding hydrochloric acid. Preferably, about 1.0-1.4 equivalents of about 32% (v:v) hydrochloric acid is added to induce precipitation. Most preferably, about 1.1 equivalents of about 32% (v:v) hydrochloric acid is added to induce precipitation. More preferably, the acidifying step is achieved at a pH about 1 to about 4. Most preferably, the acidifying step is achieved at a pH about 3.

Preferably, the washing step is achieved by using isopropanol. Preferably, about 5 to about 15 ml of isopropanol is used to wash the precipitates. Most preferably, about 10 ml of isopropanol is used to wash the precipitates.

Preferably, the crystallizing step is achieved by adding about 3 to about 5 volumes of water to induce crystallization. Most preferably, about 4 volumes of water is used to induce crystallization.

Preferably, the crystallizing step is performed in the presence of activated carbon. Preferably, the activated carbon is selected from the group consisting of SX-2, CA-1, CXV, and SX-1.

Preferably, the crystallizing step is performed in the presence of about 5 to about 15% SX-1 activated carbon. Most preferably, the crystallizing step is performed in the presence of about 10% SX-1 activated carbon.

The present invention is further explained by the following examples. The present invention is by no means restricted to these specific examples. One of ordinary skill in the art will understand how to vary the exemplified preparations to obtain the desired results.

### EXAMPLES

### Example 1:

### Preparation of Pure Dimethylamino-Methyl-Carbazolone Salt

Into 180 ml glacial acetic acid 45 gram (0.226 mole, 1.0 eq) of methyl-carbazolone, 22.4 gram (0.275 mole, 1.22 eq) of dimethylamine hydrochloride and 9 gram (0.3 mole, 1.33 eq) of paraformaldehyde were added.

The reaction was kept at about 80±2° C. during 12 hours, then 540 ml of water and 4.5 gram of highflow are introduced into the reactor, the batch was cooled to about 10° C. and rendered basic with about 45% NaOH to about pH 13 to about 14 while the batch temperature did not exceed about 25° C.

Then the batch was stirred at about 5 to about 10° C. for an additional 1 hour, the precipitate that formed along with the highflow were collected and dried in vacuum oven at about 60° C. until constant weight to obtain crude product containing highflow.

The crude product was treated with 3.3 gram activated carbon type SX-1 (by NORIT) in 990 ml acetone, filtered, cooled to about 25° C. and hydrochloric acid was bubbled through the acetone solution until pH was about 3, the batch was cooled to about 0 to about 5° C., kept at this temperature for half an hour, filtered, washed with about 20 ml acetone and dried in an oven at about 50° C. until constant weight to give 49.6 gram dimethylamino-methyl-carbozolone-HCl.

### Example 2:

### Preparation of Pure Ondansetron Base

Into 330 ml water 33 gram (0.112 mole, 1 eq.) dimethylamino-methyl-carbozolone-HCl, 3.3 gram highflow, 46.3 gram (0.563 mole, 5 eq) methyl-imidazole were added.

The reaction was heated at reflux during 12 hours and cooled to about 5 to about 10° C., the precipitate was filtered, washed with 3×300 ml water and dried in a vacuum oven at about 60° C. until constant weight to give crude compound containing highflow.

The crude compound was treated with 1.5 gram activated carbon type SX-1 (by NORIT) in 930 ml methanol, filtered (hot filtration) from the highflow and activated carbon and crystallized at 0 to about 5° C. during one hour. Hot filtration was around 60° C. and was done with methanol near its boiling point (i.e. 65° C.). The precipitate was collected by filtration, washed with 2×20 ml cold methanol and dried in vacuum oven at about 60° C. until constant weight to give 21.3 gram ondansetron-base.

### Example 3:

### Preparation of Pure Ondansetron Hydrochloride Dihydrate

Into 100 ml of water 20 gram ondansetron-base were introduced. To the stirred suspension 7.5 ml (1.1 equivalents) of about 32% hydrochloric acid (HCl) was added. A slightly exothermic reaction occurred, the suspension turned almost clear and a precipitate began to form.

The reaction was cooled down and kept at about 3-5° C. for an additional 1 hour filtered, washed, with about 10 ml cold isopropanol and dried at about 50° C. under vacuum.

### Example 4:

### Recrystallization of Ondansetron Hydrochloride Dihydrate

Ondansetron-HCl-2H₂O was crystallized twice from 1:4 w/v water and about 10% w/w activated carbon type SX-1 (by NORIT) at about 95° C. during half an hour, filtered (hot filtration), washed with 1 volume of hot water, cooled to about 5° C. and kept at this temperature for about 1 hour. The crystals was collected, washed with about 10 ml cold isopropanol and dried to give pure ondansetron-HCl-2H₂O. The obtained pure ondansetron hydrochloride dihydrate was determined by HPLC to be at least greater than 99.0%. The obtained pure ondansetron hydrochloride dihydrate contained less than 0.01% exo-methylene by-product or undetectable.

What is claimed is:

1. Ondansetron hydrochloride dihydrate having a purity of at least 99.0%.

2. Ondansetron hydrochloride dihydrate having a purity of at least 99.5%.

3. Ondansetron hydrochloride dihydrate having a purity of at least 99.9%.

4. A process for preparing dimethylamino-methyl-carbazolone comprising the steps of:
a) preparing a solution of methyl-carbazolone having the formula: 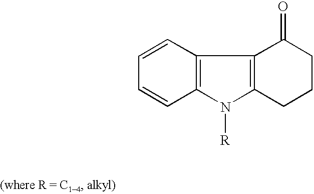
b) heating the solution in the presence of dimethylamine hydrochloride and paraformaldehyde;
c) basifying the solution to form a precipitate;
d) separating the precipitate from the solution;
e) drying the precipitate.

5. The process according to claim 4, wherein R is methyl.

6. The process according to claim 4, wherein the heating step is performed at a temperature of about 70° C. to about 100° C.

7. The process according to claim 4, wherein the heating step is performed at a temperature of about 80° C. to about 90° C.

8. The process according to claim 4, wherein the heating step is performed for about 6 to about 24 hours.

9. The process according to claim 4, wherein the heating step is performed for about 6 to about 12 hours.

10. The process according to claim 4, wherein the heating step is performed in acetic acid.

11. The process according to claim 4, wherein about one equivalent methyl-carbazolone is heated in the presence of about 1.1 to about 1.5 equivalents of dimethylamine hydrochloride and paraformaldehyde.

12. The process according to claim 4, wherein about one equivalent methyl-carbazolone is heated in the presence of about 1.2 equivalents of dimethylamine hydrochloride and formaldehyde.

13. The process according to claim 4, wherein about one equivalent methyl-carbazolone is heated in the presence of about 1.1 to about 1.5 equivalents of dimethylamine hydrochloride and formaldehyde.

14. The process according to claim 4, wherein about one equivalent methyl-carbazolone is heated in the presence of about 1.2 equivalents of dimethylamine hydrochloride and formaldehyde.

15. The process according to claim 4, wherein about one equivalent methyl-carbazolone is heated in the presence of about 4 to about 6 volumes of acetic acid.

16. The process according to claim 4, wherein about one equivalent methyl-carbazolone is heated in the presence of about 4 volumes of acetic acid.

17. The process according to claim 4, wherein the solution of methyl-carbazolone is basified by about 45% sodium hydroxide.

18. The process according to claim 17, wherein the solution is basified to a pH of about 13 to about 14.

19. The process according to claim 17 or 18, wherein the basifying step is performed in the presence of 10% celite.

20. A process for preparing ondansetron base, comprising the steps of:
a) preparing a solution of methyl-imidazole and dimethylamino-methyl-carbazolone of the formula 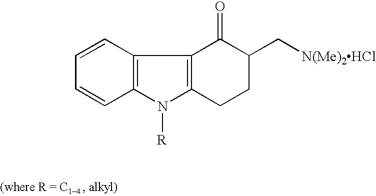
b) heating the solution;
c) removing a precipitate containing ondasetron base from the solution;
d) washing the precipitate;
e) drying precipitate to obtain ondansetron base.

21. The process according to claim 20, wherein the solution is prepared by adding about 4 to about 6 equivalents methyl-imidazole to one equivalent dimethylamino-methyl-carbazolone.

22. The process according to claim 20, wherein the solution is prepared by adding about 5 equivalents methyl-imidazole to one equivalent dimethylamino-methyl-carbazolone.

23. The process according to claim 20, wherein the solution is prepared in the presence of 10% celite.

24. The process according to claim 20, further comprising the step of:
recrystallizing ondansetron base.

25. The process according to claim 24, wherein the recrystallizing step is performed in the presence of activated carbon and methanol.

26. A process of preparing pure ondansetron hydrochloride dihydrate comprising the steps of:
a) preparing a solution of ondansetron base;
b) acidifying the solution with hydrogen chloride to form a precipitate;
c) washing the precipitate; and
d) crystallizing pure ondansetron hydrochloride dihydrate.

27. The process according to claim 26 wherein about 3 to about 7 volumes of water is added to ondansetron base to prepare a solution of ondansetron base.

28. The process according to claim 26 wherein about 5 volumes of water is added to ondansetron base to prepare a solution of ondansetron base.

29. The process according to claim 26 wherein about 1.0 to about 1.4 equivalents of about 32% (v:v) hydrochloric acid is added to acidify the solution to induce precipitation.

30. The process according to claim 26 wherein about 1.1 equivalents of about 32% (v:v) hydrochloric acid is added to acidify the solution to induce precipitation.

31. The process of claims 29 or 30, wherein the solution is acidified to a pH about 1 to about 4.

32. The process of claims 29 or 30, wherein the solution is acidified to a pH about 3.

33. The process according to claim 26, wherein the precipitate is washed with about 5 to about 15 ml of isopropanol.

34. The process according to claim 26, wherein the precipitate is washed with about 10 ml of isopropanol.

35. The process according to claim 26, wherein the crystallizing step is achieved by adding about 3 to about 5 volumes of water to induce crystallization.

36. The process according to claim 26, wherein the crystallizing step is achieved by adding about 4 volumes of water to induce crystallization.

37. The process according to claim 26, wherein the crystallization step is repeated two times.

38. The process according to claim 26, wherein the crystallizing step is achieved in the presence of activated carbon.

39. The process according to claim 36, wherein the activated carbon is selected from the group consisting of SX-2, CA-1, CXV and SX-1.

40. The process according to claim 39, wherein the activated carbon is about 5 to about 15% SX-1.

41. The process according to claim 39, wherein the activated carbon is about 5 to about 10% SX-1.

42. Ondansetron hydrochloride dihydrate as prepared in accordance with a process of claim 26, wherein the ondansetron hydrochloride dihydrate has a purity of at least about 99.0%.

43. Ondansetron hydrochloride dihydrate as prepared in accordance with a process of claim 26, wherein the ondansetron hydrochloride dihydrate have a purity of at least about 99.5%.

44. Ondansetron hydrochloride dihydrate as prepared in accordance with a process of claim 26, wherein the ondansetron hydrochloride dihydrate has a purity of at least about 99.9%.

45. A pharmaceutical formulation comprising ondansetron hydrochloride dihydrate as prepared in accordance with a process of claim 26, wherein the ondansetron hydrochloride dihydrate has a purity of at least about 99.0%.

46. A pharmaceutical formulation comprising ondansetron hydrochloride dihydrate as prepared in accordance with a process of claim 26, wherein the ondansetron hydrochloride dihydrate has a purity of at least about 99.5%.

47. A pharmaceutical formulation comprising ondansetron hydrochloride dihydrate as prepared in accordance with a process of claim 26, wherein the ondansetron hydrochloride dihydrate has a purity of at least about 99.9%.
